(19) United States
(12) Patent Application Publication
(10) Pub. No.: US 2024/0350199 A1
(43) Pub. Date: Oct. 24, 2024

(54) METHODS, SYSTEM AND APPARATUSES FOR THE INDUCTION AND REGULATION OF IMMUNOLOGICAL RESPONSES BY TISSUE ABLATION VIA PERMEABILIZATION OF CELL MEMBRANES AND ELECTROLYSIS

(71) Applicant: Inter Science GmbH, Gisikon (CH)

(72) Inventors: Enric Robert Guenther, Bouveret (CH); Michael Klaus Stehling, Roedermark (DE)

(21) Appl. No.: 18/641,059

(22) Filed: Apr. 19, 2024

Related U.S. Application Data
(60) Provisional application No. 63/460,904, filed on Apr. 21, 2023.

(51) Int. Cl. A61B 34/10; A61B 18/00; A61B 18/02; A61B 18/12; A61B 18/14

(52) U.S. Cl. CPC A61B 34/10; A61B 18/02; A61B 18/1206; A61B 18/14; A61B 2018/00005; A61B 2018/00577; A61B 2018/00648; A61B 2018/00714; A61B 2018/0072; A61B 2018/00761; A61B 2018/00767; A61B 2018/00791; A61B 2018/00875; A61B 2018/00994; A61B 2034/104; A61B 2034/107

(57) ABSTRACT

Examples of systems and procedures for tissue ablation and the induction of immunological responses are described. Examples combine electroporation-a technique for creating temporary openings in cell membranes through electrical pulses (or other cell permeabilization techniques)—with electrolysis, a process that chemically alters tissues via electric current (“E2”). This combination is utilized to selectively target and ablate cancerous tissues while concurrently stimulating an immunological response allowing a wide range of parameter choices, potentially enhancing the efficacy of cancer treatments.

### CROSS-REFERENCE TO RELATED APPLICATIONS

This application claims priority to U.S. Provisional Application Prov. No. 63/460,904 filed Apr. 21, 2023, the contents of which are incorporated herein by reference, in their entirety, for any purpose.

### FIELD

The present invention relates to medical devices and therapeutic methods, and more particularly to systems and procedures for tissue ablation and the induction of immunological responses. Specifically, the invention pertains to the field of oncology and surgical procedures involving the ablation of neoplastic tissues. It encompasses an integrated approach that combines electroporation-a technique for creating temporary openings in cell membranes through electrical pulses (or other cell permeabilization techniques)—with electrolysis, a process that chemically alters tissues via electric current (“E2”). This combination is utilized to selectively target and ablate cancerous tissues while concurrently stimulating an immunological response allowing a wide range of parameter choices, potentially enhancing the efficacy of cancer treatments. The invention falls under the categories of electrochemical therapy, cancer immunotherapy, and minimally invasive surgical technologies.

### BACKGROUND OF THE INVENTION

Immunotherapy has emerged as a transformative approach for cancer treatment, involving the activation or suppression of the immune system. Modalities such as immunomodulation therapies (interleukins, cytokines, chemokines, IMiDs), activation/enhancement immunotherapies dendritic cell activation, T-Cell transfer), and checkpoint inhibitors (MABs, e.g. CTLA-4, PD-1, PD-L1) have shown promise. However, their effectiveness is often limited by the ability of malignant neoplasms to evade immune detection, facilitated by immune-permissive microenvironments and other tumor evasion mechanisms.

One strategy to counteract the immunosuppressive tumor microenvironment is the induction of cell death through radiation or tissue ablation, which can lead to systemic immune responses. These responses may be augmented by immunotherapies, making local tumor ablation an attractive adjunctive treatment. Minimally invasive tissue ablation techniques include thermal and non-thermal applications, utilizing electricity. Thermal modalities involve raising or lowering tissue temperature, while non-thermal techniques like electrolysis and electroporation affect cells within the tissue without damaging the extracellular matrix or other structures.

Electrolytic ablation, used since the early 1800s, employs electric currents to generate cytotoxic environments that induce cell death through pH changes. Although advantageous due to low voltage and current requirements, electrolytic ablation suffers from long treatment durations and unpredictable distribution of electrolytic products, making ablation dimensions difficult to predict. Electrolytic ablation alone has been reported to induce immunological responses for cancer treatment.

Electroporation, on the other hand, permeabilizes cell membranes using pulsed electric fields. Lower electric fields produce reversible electroporation (RE), while higher fields result in irreversible electroporation (IRE) and cell death. The quick and non-thermal nature of IRE, alongside its ability to spare sensitive structures, has positioned it as a favorable option in tumor ablation. Despite these advantages, the application of IRE is not without challenges, as it necessitates high electric fields and can induce muscle contractions and electrode displacement due to the required pulse intensity. IRE alone has been reported to induce immunological responses for cancer treatment as well.

The electrolytic electroporation (E2) method combines electroporation and electrolysis, offering a more efficient and adjustable tissue ablation technique with fewer pulses and lower electric fields than conventional IRE. In prior disclosures, including patents No. US20210186592A1 and No. US20210330371A1, the expansive parameter space of the referenced base patent US10390874B2 was harnessed to improve the efficiency, safety, and speed of tissue ablation using E2. The current invention builds upon this foundation, elucidating the application of E2 with a specific focus on immunological reactions. By leveraging the adjustable parameters of E2, it is possible to initiate diverse cell death pathways, which in turn can trigger various immunological responses necessary for the effective treatment of cancer, making previously ‘invisible’ tumors more recognizable to the immune system.

### SUMMARY OF THE INVENTION

In fine tuning the parameters for tissue ablation (initial electric potential, wave shape and electrode placement amongst others), and thereby inducing certain voltage intensities at designated locations within the targeted tissue, different cell death modes can deliberately be induced, initiating or modulating different immune reactions associated with different cell death modes. This can be used as a standalone therapy or as a complementary treatment with existing immunotherapies. Notably, the majority of these adjustments, excluding those related to the type of applicator, can be implemented through software configuration and strategic placement of the previously established E2 delivery system without necessitating physical alterations to the equipment. In other words, it allows inducing all known ablation induced cell death modes to a volume or volumes of interest by just the system setting the right parameters.

Amongst the cell death modes achievable through the fine-tuning of E2 parameters, are heat-based, acid/base-based, pH-based disruption of the tumor micro environment (TME) and ferroptosis, in addition to those offered by “pure” electroporation based ablation (apoptosis, necrosis, autophagy, and necroptosis and pyroptosis depending on the specific application and parameters used). This expanded range of cell death modes, in addition to the incited release of heat shock proteins (HSPs) associated with RE and certain cell death modes, enables E2 to stimulate both the innate and adaptive immune systems. The adaptability of E2, combined with its ability to deliver therapeutic agents, such as small molecules, gene therapy vectors, cytotoxic substances, or cytokines, through electroporation, makes it an ideal and near universal candidate for use in conjunction with immunotherapies.

The exact designation of treatment parameters, including those established in prior disclosures, can be tailored by clinicians for the desired treatment outcome and based on factors such as cancer or tissue type, desired therapy endpoint, the comprehensive treatment plan, and any accompanying therapies, whether immune-based, chemotherapeutic, or other. The significance of such customization is evident within current therapeutic paradigms and is anticipated to become increasingly important as medical practice advances towards more personalized treatment approaches for cancer.

An example method according to principles of the present disclosure may include applying a pulse of at least one of a voltage or a current to an ablation target encompassed in one or more electrodes, wherein the pulse is configured to cause electroporation and electrolysis at the ablation target and modulating the pulse to achieve certain voltage densities (voltage per unit area) which promote the desired cell death modes at a specific location (e.g. large penumbra of necroptosis with several small areas of thermal necrosis and other areas with chemical necrosis) in the tissue/tumor and cause a predetermined immune reaction.

### BRIEF DESCRIPTION OF DRAWINGS

The provided illustrations depict various aspects of embodiments of the current invention. However, it is important to note that these drawings are not intended to impose limitations or rigidly define the invention. Rather, in conjunction with the accompanying written description, they aim to elucidate specific principles underlying the invention.

FIG. 1 is a block diagram of a system for delivering electrolytic electroporation according to an embodiment of the disclosure.

FIG. 2 is a table associating different E2 obtainable ablative energy (e.g. field strength) with a more extensive description of the cell death modes and their associated immunological reactions.

FIG. 3A is a diagram depicting electric field intensity between two plate electrodes in an axial cut plane.

FIG. 3B is a diagram depicting electric field intensity between two pairs of 3 needle electrodes in an axial cut plane.

FIG. 4A is a diagram depicting a side view of the mathematically modelled electrical field intensity and associated cell-death modes for a specific applicator geometry and tissue properties.

FIG. 4B is a diagram depicting a top view of the mathematically modelled electrical field intensity and associated cell-death modes for a specific applicator geometry and tissue properties.

FIG. 5 is a graph depicting the shape of a typical electroporation-based ablation pulse sequence (typically few short [microseconds], high voltage [thousands of Volt] pulses).

FIG. 6 is a graph depicting the shape a typical electrolysis-based ablation pulse sequence (typically long [minutes], low voltage DC current).

FIG. 7 is a graph depicting the shape of a typical E2 heat-based ablation pulse sequence (typically 100 kHz to few GHz low voltage pulses [radio frequency or microwave]).

FIG. 8 is a graph depicting the shape of a typical E2 pulse sequence incorporating all electric-based types of ablation in one exponential decay (e.g. capacitor discharge typically lasting tens of milliseconds to few seconds).

FIG. 9A is a graph depicting the sequence of an E2 application with non-electrical poration followed by electrolysis.

FIG. 9B is a graph depicting the shape of a typical E2 decreasing chopped pulse (e.g. to reduce tissue heating and increase the electroporation part of the ablation).

FIG. 9C is a graph depicting the shape of a typical E2 decreasing chopped pulse followed by electrolysis delivery via low voltage DC current.

FIG. 9D is a graph depicting the shape of a typical E2 exponential decay chopped pulse followed by heating.

FIG. 10 is a graph depicting the shape of a typical E2 combined sequence with bipolar pulses for example (e.g. to increase electroporation and decrease the electrolysis distribution).

FIG. 11A is an image of an E2 ablation site with two needle electrodes in pig liver using a low energy, heavily electroporation-based pulse (no thermal or chemical necrosis).

FIG. 11B is a schematic outlining the visible cell death modes from the ablation in FIG. 11A.

FIG. 12A is an image of an E2 ablation site with two needle electrodes in pig liver using a pulse with double the total electric energy as for that of FIG. 11, but still heavily electroporation based (some thermal and chemical necrosis in close proximity to the electrodes visible but large death due to electroporation combined with electrolysis [necroptosis]).

FIG. 12B is a schematic outlining the visible cell death modes from the ablation in FIG. 12A.

FIG. 13A is an image of an E2 ablation site with two needle electrodes in pig liver using a higher amount of energy than for FIG. 11-12 showing significant amount of necrosis inside the ablation penumbra also yielding the highest immune response in terms of macrophage count (ref. FIG. 15).

FIG. 13B is a schematic outlining the visible cell death modes from the ablation in FIG. 13A.

FIG. 14 is an example H&E slide at 20× from a study on healthy pig liver using a needle insertion analogous to FIG. 13. Black dots show macrophage invasion 24 h post ablation which was used to quantify the immune responses for different sequences exemplary shown in FIG. 15.

FIG. 15 is a diagram depicting the relative area (quantity) of macrophages for different methods of energy delivery. Simplified but efficiently it shows that a combination of different choices of parameters will lead to modes of cell death which in turn can be used to modify amplitude and type of immune response which is claimed here in form of a system, apparatus, and method.

### DETAILED DESCRIPTION OF THE INVENTION

Certain details are set forth below to provide a sufficient understanding of embodiments of the disclosure. However, it will be clear to one skilled in the art that embodiments of the disclosure may be practiced without these particular details. Moreover, the particular embodiments of the present disclosure described herein are provided by way of example and should not be used to limit the scope of the invention to these particular embodiments. In other instances, well-known materials, components, processes, controller components, software, circuitry, timing diagrams, and/or anatomy have not been described or shown in detail in order to avoid unnecessarily obscuring the embodiments.

Examples of technology described herein may be related to and/or used with technology described in U.S. Patent Application Publication No. U.S. Pat. No. 10,390,874B2, No. US20210186592A1 and No. US20210330371A1, all of which are incorporated herein by reference in their entirety. These referenced applications provide foundational technology and methodologies upon which the current invention elaborates and improves. This application seeks to advance the technological field established by these earlier applications by introducing innovative enhancements and refinements as detailed herein.

Cancer Immunology is an important subject in the field of oncology. Several immunological approaches for the treatment of cancer already exist and new methods are being discovered at an increasing pace.

However, core problems to all approaches (MABs/checkpoint inhibitors, dendritic cell or t-cell therapy, natural killer cells, etc.) remain, amongst others the tumor microenvironment, which is usually highly anti-inflammatory and the lack of suitable antigen presentation mechanisms to the immune system, rendering some tumors “immunologically silent”. Also, macroscopic sizes of tumors (everything above millimeter sizes) pose an inherent challenge for the immune system to battle even if recognized (with or without treatments) as thread. Efforts are being undertaken to find the optimal methods to solve these problems for different types of cancer.

Local tumor site treatment offers solutions. Radiation therapy and ablative therapies are well suited in principle to induce immune reactions directed against the cancer but have different shortcomings.

Irreversible Electroporation (IRE) based tissue ablation, among the ablative therapies, has a high potential to become a method of choice for this need. Its fundamental mechanism induces a very robust mix of several cell death modes which appears to be well suited for this need. However, the parameter space of IRE, especially as described in its original patent (U.S. Pat. No. 8,048,067B2) is very limited as it does not respect the production and use of electrolysis which often is core part of the induced cell death mechanism and is by design non-thermal.

It was found that E2 is an improved and more flexible method to induce cell death by means of combining electroporation and electrolysis, is well suited to induce designed immunological responses because it offers a wider parameter range and can induce additional signals like heat shock protein and various cell death modes. Also, E2 is less constrained than IRE for the use of large volume ablations and complex geometries which often occur in context with advanced cancers.

It is therefore proposed that E2, electroporation (permeabilization) and electrolysis, be used as an in vivo immuno-stimulant to induce immunological mechanisms which can be used as a treatment for neoplasms, both benign and malignant, such as cancers, sarcomas, lymphomas, and any other. This initial immune stimulation with E2 can be used to make “immunologically silent” tumors visible to the immune system similar to IRE and other electroporation and physio-chemical ablation modalities and thus amenable to immunotherapy and/or to improve the response of all tumors to immunotherapy and/or as a stand-alone ablation for curative or palliative purposes with the idea for a systemic immune response to potentially residual or spread tumor cells. E2 is further uniquely ideal for this purpose as a volume of interest can be designed to have a plethora of different cell death modes and subsequent immune response while simultaneously being suited to almost any geometry of delivery electrodes. Therefor we show and claim the novelty to use it as an “universal ablation tool” for designing fast and simple and safe ablation procedures with adjustable ablation mode (and subsequent response types).

Many existing types of ablation cause an immune response. Ablation techniques can be categorized into thermal and non-thermal, thermal techniques using either temperature increase or decrease, to cause cell death. radiofrequency ablation (RFA), microwave ablation (MWA), and high-intensity focused ultrasound (HIFU) do the latter. Although the heat leads to protein denaturation and coagulative necrosis, mechanisms by which tumor antigens and damage-associated molecular patterns (DAMPs) can be released, stimulating an immune response, the intense heat often destroys immune cells and denatures proteins but importantly also destroys all micro and macro vasculature of the treated volume (tumor) reducing the potential effectiveness of said response.

Likewise, cryoablation uses extreme cold to achieve cell destruction, and while it can preserve antigens better than heat-based methods, potentially leading to a more pronounced immune response, it shares some limitations. The process can also cause significant cell destruction beyond the targeted area, leading to local inflammation that may not be conducive to a balanced immune response. Additionally, the larger ice crystals formed during the freezing process can rupture blood vessels, impeding the oxygen supply, which is vital for a sustained immune attack, and can result in avascular necrotic zones that limit immune cell access. Electrolysis and various modes of electroporation rely on non-thermal mechanisms of tissue ablation, where they affect only the cells within tissue, sparing the extracellular matrix and other organ structures. Several applications are advantageous for non-thermal ablation, in particular treatment of tumors which are in high proximity to sensitive sites.

In electrolytic ablation, electric currents are delivered through two electrodes which encompass the targeted tissue. The current is delivered in such a way as to produce electrolysis at the surface of electrodes submerged in tissue, which is an ionic conducting media. New chemical species such as hypochlorous acid (HCIO) are generated at the interface of the electrodes and diffuse away from the electrodes into the tissue. This diffusion occurs along a concentration gradient and by electrophoresis. These species are able to create a cytotoxic environment which can induce cell death, a leading mechanism being local changes in pH, with other mechanisms also possibly at play. Both, the cytotoxic environment, and pH changes lead to the release of various intracellular contents, disrupt the TME, including DAMPs, which can recruit and activate immune cells to the site of ablation, further assisting in the body's natural anti-tumor response and potentially enhancing the efficacy of subsequent immunotherapies. The low voltage and current requirements of electrolytic ablation stand as advantages over other techniques, offering simplicity in apparatus design. Nevertheless, the process demands a high concentration of electrolytic products, entailing lengthy treatment times (tens of minutes to hours), which is a drawback. Additionally, the long treatment time facilitates normal, non-Nernst-Planck type diffusion and blood transportation phenomenon. This can lead to an almost unpredictable distribution of electrolytic products and therefore difficult predictability of ablation dimensions.

Electroporation is the permeabilization of the cell membrane by a pulsed electric field delivered across the cell. The effect on the cell membrane is a function of the electric field strength and pulse time duration. Lower electric fields produce reversible electroporation, in which case the cell returns to its original state a few seconds or minutes after the electric field has ceased. This phenomenon is used for gene delivery, uptake of drugs or genetic material into cells, inserting proteins into the cell membrane, and fusing between individual cells. Electrochemotherapy, the combination of reversible electroporation and chemotherapeutical drugs, such as bleomycin, has been used successfully for tumor ablation in clinical settings. The reversible permeabilization of cell membranes not only allows for the uptake of therapeutic substances but also exposes intracellular antigens to the immune system, which can provoke an immune response against cancer cells, particularly when combined with immunomodulatory drugs. Electrochemotherapy usually utilizes eight 100 microsecond long pulses, with electric fields of between 200-500 V/cm (1000-1500 V/cm voltage to needle type electrode distance ratio). While the application is effective at cancer treatment, it requires the application of drugs, putting it into the regulatory domain of drug therapies.

A combination of higher electric fields and longer exposure time of these electroporation pulses results in cell death through a mechanism broadly referred to as irreversible electroporation (IRE), i.e. the cells succumb to the membrane permeabilization by electroporation. IRE has gained success in clinical tumor ablation. IRE can ablate tissues without the need for drug injection and without resorting to thermal damage. This is why the procedure is also known as non-thermal irreversible electroporation (NTIRE). The NTIRE procedure is much faster than conventional electrolytic ablation and preserves sensitive structures. IRE's ability to preserve sensitive structures, such as blood vessels, means that the immune system's access to the ablated area is maintained. This preservation facilitates the infiltration of immune cells, such as T-cells and macrophages, and can initiate a stronger immune reaction against remaining cancer cells. However, the procedure employs very high electric fields in the order of 500-1000 V/cm (1500 to 3000 V/cm voltage to needle type electrode distance ratio) and sometimes hundreds of pulses over minutes with strict limitations on distance and parallelism of electrodes. The use of high electric fields and the large number of pulses used in NTIRE has disadvantages. NTIRE pulses induce muscle contractions that require the use of a muscle relaxant and deep anesthesia during surgery. The muscle contractions may also move the electrodes during treatment, resulting in possible complications. This is particularly detrimental when hundreds of pulses are delivered. Additionally, the high fields almost inevitably produce a high amount of electrolytic products and spark plasma (after some pulses) causing a pressure wave (referred to as discharges or sparks or arcing) with loud acoustic manifestation and mechanical tissue damage. An example numerical value for electric fields that develop across a gas layer to generate an electric breakdown and the consequent sparks may be approximately 30 kV/cm in some applications. Technically, even if stopped in time, they can cause low-impedance situations which will cause machine failures and with that a risk for the patient. In addition, while the actual electroporation part of the procedure is brief, the logistic complications associated with the placement of the electrodes and the large number of pulses substantially lengthen the procedure.

Electrolytic electroporation (E2) ablation technology, the combination of electrolysis and electroporation, upon which the present invention builds, was developed in a systematic way, from basic concept through small animal studies to large animal studies. E2 may provide a minimally invasive tissue ablation technology with advantages over tissue ablation by either electroporation or electrolysis alone. One potential advantage is that E2 requires substantially fewer electric pulses and at a lower electric field than conventional NTIRE, thereby avoiding the challenging limits of NTIRE such as maximum ablation size per electrode placement and electrical safety design. This reduced reliance on high-intensity pulses also makes E2 particularly well-suited for the complex geometries often encountered in advanced cancers, where maintaining the integrity of the surrounding anatomy is critical. Additionally, E2 is non-thermal and does not require the injection of drugs, unlike Electrochemotherapy, which requires the injection of bleomycin or other agents. Without being bound to a particular theory, a mechanistic explanation of the E2 technology may be related to the permeabilization of the cell membrane by all modes of electroporation and nano pulses. The products of electrolysis may, thereby, gain access to the interior of the cell by the electroporation permeabilized cell membrane (homeostasis impairment), and cause cell death at a much lower dose than that required for tissue ablation by conventional electrolysis.

The E2 method always includes an applicator which is in contact with tissue. The surface of the applicator is the place where the electrolytic species are formed by electrochemical reactions between the charged surface and the tissue, usually but not necessarily by means of DC current between an anode and a cathode. Importantly the form and shape of the applicator are not limited to a needle, which is the most common design. E2 applicators can also take the form of surface pads, skin applicators, surgical tools that can be added to endoscopes, or other similar devices. Also, the electrolysis production in E2 is not limited to a specific amount, method, or a specific metal type of the surface. This flexibility allows for a wide range of possible electrolytic reactions that can be designed and chosen depending on the specific requirements of the treatment.

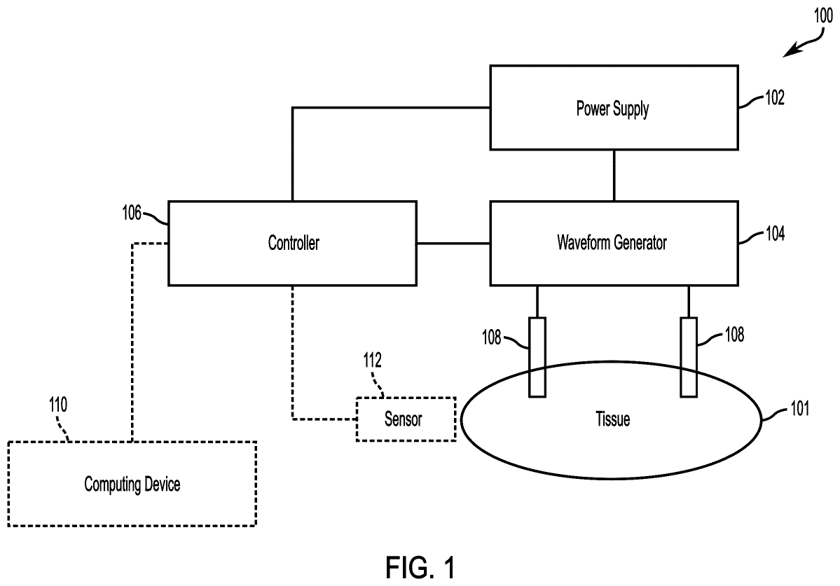

FIG. 1 is a block diagram of a system 100 for delivering electrolytic electroporation (E2) according to principles of the present disclosure. In some embodiments, the system 100 may include a power supply 102, a wave form generator 104, a controller 106, and one or more electrodes 108. For context, an ablation target is also shown. In the example in FIG. 1, tissue 101 is the ablation target.

The power supply 102 may provide a programmed current and/or voltage to the waveform generator 104 and/or controller 106. In some embodiments, the power supply 102 may not be directly coupled to the waveform generator 104 and power is supplied from the power supply 102 to the waveform generator 104 via the controller 106. In some embodiments, the controller 106 may selectively couple and decouple the power supply 102 from the waveform generator 104. In some embodiments, a capacitor array can be part of the waveform generator 104 or be serial with the power supply 102 and the waveform generator 104.

Flexibility in applicator design, electrolysis production, and permeabilization techniques, not only circumvents the limitations of many traditional ablation methods but affords electrolytic ablation by permeabilization (and E2 more specifically) an unprecedented level of control to cause different modes of ablation and cell death to a volume of interest simply by choice of energy parameters which can entirely be set, calculated and controlled by software. Hence E2's parameters, as subsequently outlined, can be tailored on a case-by-case basis, in order to induce such immunological reactions deemed to be most beneficial for the patient, taking into consideration cancer or tissue type, desired therapy endpoint, the comprehensive treatment plan, and any accompanying therapies.

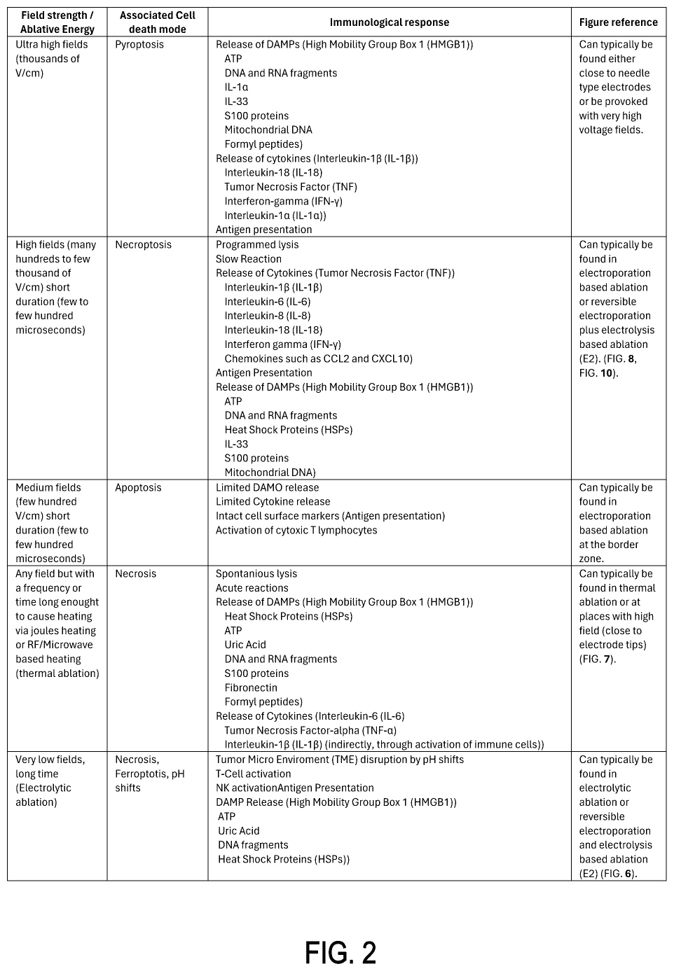

It has been studied, that E2 ablation under different operational parameters is able to achieve almost any combination of known cell death types by ablation, each known to be associated with different immunological responses by the organism. The spectrum of immunological outcomes from different cell death modalities-including but not limited to apoptosis, pyroptosis, necroptosis, necrosis and ferroptosis-encompasses a range of inflammatory responses and the release of various signaling molecules. These processes, depending on their nature and intensity, can elicit a variety of immune reactions. Some of these reactions involve the immune system's uptake of cellular debris, the generation of DAMPs, the alteration of the tumor microenvironment from immunosuppressive to immunopermissive, as well as the upregulation of T-cell production and enhanced invasion of macrophages into tumor sites. FIG. 2 is a table associating different field strengths, achievable under E2 with a more extensive description of the cell death modes and their associated immunological reactions.

A key advantage of methods like IRE and, more notably, E2, lies in their ability to preserve larger blood vessels post-ablation. This preservation is vital as it supports a more robust influx of immune cells into the ablated area, facilitating a more dynamic and potentially effective immune response compared to other ablation techniques.

The present disclosure describes a method by which immunological reactions can specifically be targeted in a patient based on modelling predictions and a growing database of empirical data for different electrode configurations, tissue types and pulse parameters achievable with E2. In the subsequent section the modelling methodologies for different parameters are described.

Electric field strength within any geometric configuration is determined by solving the Poisson equation ∇²φ=−ρ/ε, where φ represents the electric potential, ρ is the charge density, and ε denotes the permittivity of the medium (in some instances solving the Laplace equation suffices). Finite element modeling is utilized for these calculations, with tissue impedance monitoring potentially updating parameters in real time to enhance model accuracy.

Heat distribution within the treated tissues is calculated using the Penne's bioheat equation. This analysis, facilitated by finite element modeling, incorporates both generated heat and biological heat transfer processes such as blood perfusion. Real-time impedance measurements may be used to dynamically adjust thermal parameters.

The generation and distribution of electrolytic species are predicted through the Nernst-Planck diffusion equation. This formulation describes the migration of ions in response to electric fields and concentration gradients. Finite element models are employed for these predictions, and tissue impedance monitoring may adjust these parameters in real time.

Comprehensive computational models can simulate these physical phenomena concurrently, allowing for said holistic optimization of the ablation process based on clinical objectives.

The optimization (i.e. achievement of an immunological response optimal to the overall [immuno-] oncological treatment plan) of the ablation process in order to target and achieve certain cell death modes and in turn certain immunological reactions (also referred to as “optimizing the immune response” for the purpose of the present disclosure) involves two primary factors: the geometry of the electrodes and the design of the pulse sequence. These elements are critical in determining the distribution of the electric field, heat distribution and electrolytic species and thus consequent therapeutic outcomes.

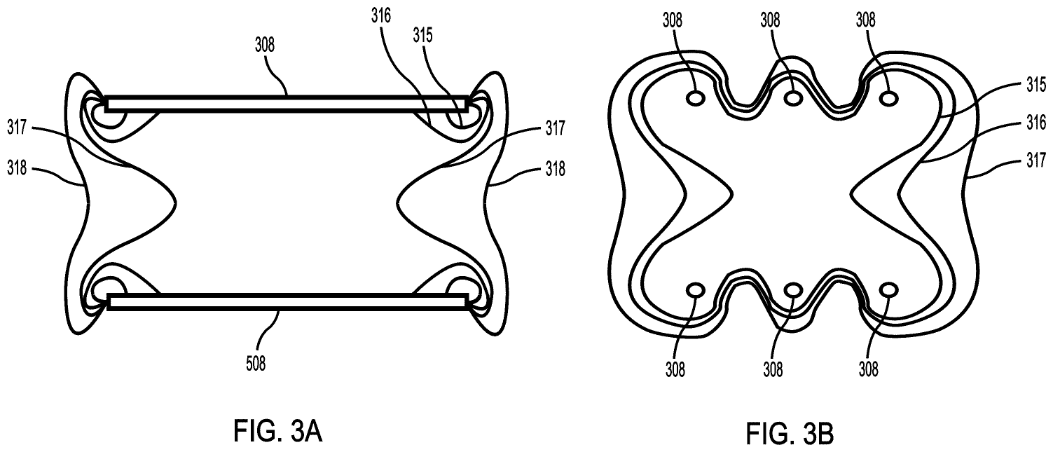

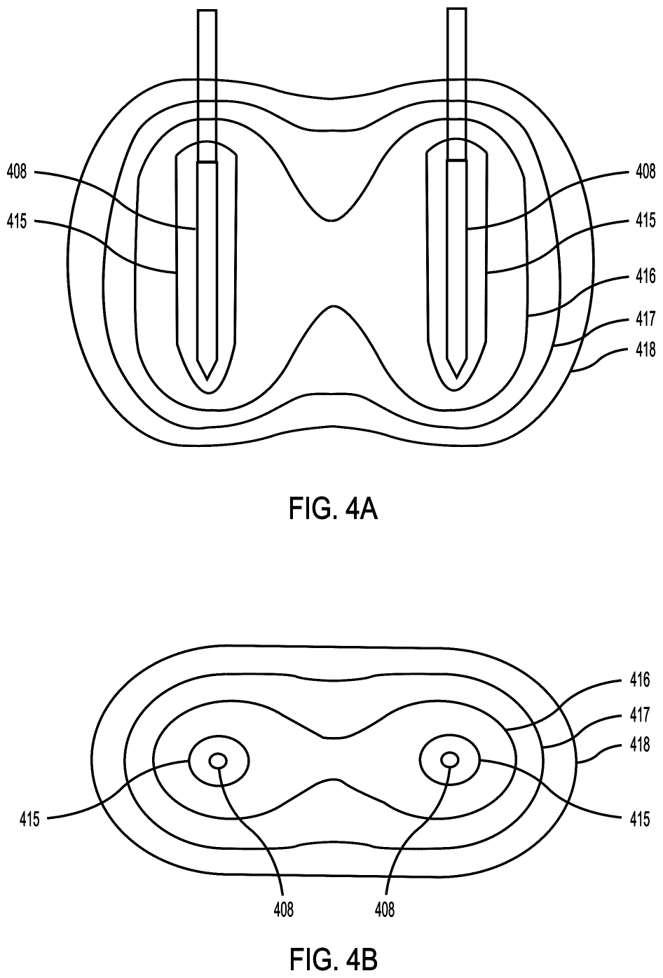

Various electrode configurations can be employed to optimize therapeutic effects. Configurations might include parallel alignments for uniform fields (FIG. 3A), or arrays for focused fields (FIG. 3B), plate or pinch electrodes (FIG. 3A), monopolar or bipolar needle electrodes (FIG. 4), needle pads, flat or curved tips and others—each tailored to specific anatomical and therapeutic needs.

FIG. 3A provides an example for the uniform electric field strength induced by two parallel plates, whereby 315, 316, 317 and 318 are isolines indicating areas of high to lower electric intensity in the respective order. Modifying the pulse configuration allows for a precise control of field strength and subsequently electrical current, heating and electrolysis production and driven diffusion, allowing for the adjustment of just one type of cell death and hence immunological response in a specific volume. The possibility of inserting two, or just one, plate(s) in percutaneous applications is, however, unfavorable but example usage could be surgical clamps on an endoscope.

FIG. 3B provides another example for the relatively uniform electric field strength induced by a configuration of two sets of parallel electrodes 308, consisting of three adjacent needle electrodes (whereby distance between each electrode in a row is less than the distance between rows) placed on either side of a tumor, approximating the uniform electric field induced by two parallel plates with areas of high to low intensity 315, 316, 317. The field strength in between both rows, for any given pulse configuration in which all electrodes of a single row are configured to have the same polarity, is approximately uniform meaning that for most of the targeted tissue the same type of cell death and hence immunological response will occur.

Whilst the aforementioned electrode configurations are conducive to certain clinical outcomes, a configuration using a pair of needle or point electrodes is in many cases still the safest. FIG. 4 outlines a more specific computer model (based on the solution of the Laplace equation for a certain tissue impedance, electrode configuration and pulse intensity) of the electric field achieved through the placement of two needle electrodes. As an example, the electrodes themselves could be inserted into two predetermined points within the tumor geometry and through the application of a chosen pulse/pulse sequence most of the tumor, up to its margins ablated by electroporation associated cell death modes, whilst thermally ablating the areas closest to the electrodes, thereby attracting DAMPs, heat shock proteins and inducing an immunological response typically associated with electrolysis in the thermally ablated regions.

As shown in FIG. 4, the geometry of the applicators 408 together with the resulting electrical field shape can be used to regulate the prevalent cell death types, whereby areas of different peak electric intensity were found to correlate to different cell death modes (necrosis where >3000V/cm 415, necroptosis where 1000-3000V/cm 416, apoptosis where 600-1000V/cm 417 and reversible EP where 400-600V/cm 418]. But additionally, similar to the field strengths shown here, the distribution, amount and types of electrolytic species can be controlled, as well as the volumetric distribution of tissue heating. The ability to control electrical fields (electroporation strength) and electrolysis production and distribution and heat in one system is unique to E2 making it uniquely suited to produce any desired form of cell death, DAMPs and the according immunological response patterns. This approach allows clinicians to predict the geometric distribution of different cell death modes for different pulse shape designs and hence target specific immune responses in the patient.

Pulse sequences can also be adjusted to target certain immunological outcomes for given electrode configurations. First, it must be understood that there are three principal ablation modes within the parameter space of E2, consisting of electroporation (or other cell permeabilization methods like freezing or sonoporation), electrolysis, and thermal ablation, often produced alongside one another in the discharge of a single pulse. By adjusting pulse parameters (including but not limited to amplitude, pulse length, pulse polarity, pause times, total charge, total duration, rise times, fall times, overall shape, modifications to exponential decays) certain of the aforementioned ablation modes can be accentuated, thereby targeting associated cell death modes and in turn certain immunological responses.

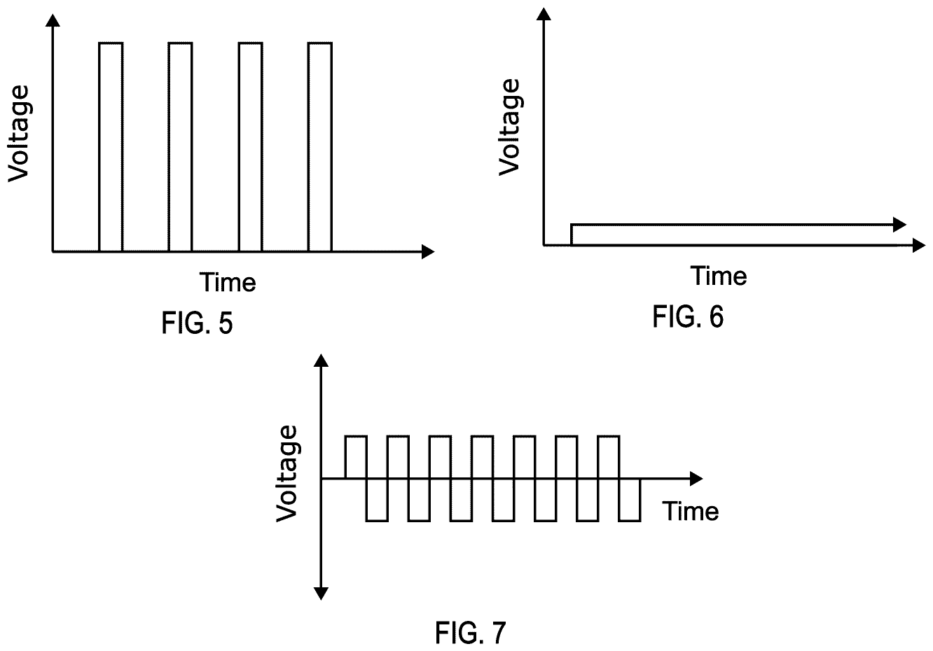

FIG. 5 outlines an E2 electroporation-based ablation optimized pulse sequence. A relatively high voltage (usually in the hundreds to thousands of V/cm) is chosen and few and short pulses (usually 4 to a few hundred with 1 to 100 us length each) are employed. Electroporation, depending on the choice of parameters will usually lead to apoptosis with limited DAMP and cytokine release but well intact surface markers and necroptosis with the release of DAMPs, and a cytokine and antigen presentation.

FIG. 6 outlines an E2 electrolysis-based ablation optimized pulse sequence. A long low voltage (usually below 48V) dc voltage is applied between two electrodes (needles or plates or needle and ground pad). This is known to cause pH shifts that disrupts the Tumor Microenvironment and activate macrophages. Ferroptosis with associated DAMP (HMGB1, ATP and uric acid and Heat shock protein release) and Cytokine Release (IL1, IL6, TND) have also been observed. Additionally increased quantities of T lymphocytes and natural killer cells have also been observed, especially near the cathode where the pH level is increased.

FIG. 7 outlines a typical pulse shape for an E2 thermal-based ablation pulse sequence. The most well-known thermal ablation techniques are heating via radiofrequency or microwave frequency at relatively low voltage fields. The associated cell death modes induce spontaneous lysis, acute reactions, and release of DAMPs (high Mobility Group Box 1 [HMGB1], heat shock proteins [HSPs], ATP, uric acid, DNA and DNA fragments, S100 proteins, fibronectin, formyl peptides, release of cytokines [interleukin-6 {IL-6}], tumor necrosis factor-alpha [TNF-α], interleukin-1B [IL-1β]) indirectly, through activation of immune cells. However, the areas of thermal necrosis are also impermeable to blood supply, therefore there is a high immunological reaction but usually little surface area for immune cells. Typical cell death modes are pyroptosis, causing the release of DAMPs, cytokines and antigen presentation and necrosis causing acute reactions, macrophage attraction, heat shock protein release and the release of DAMPS.

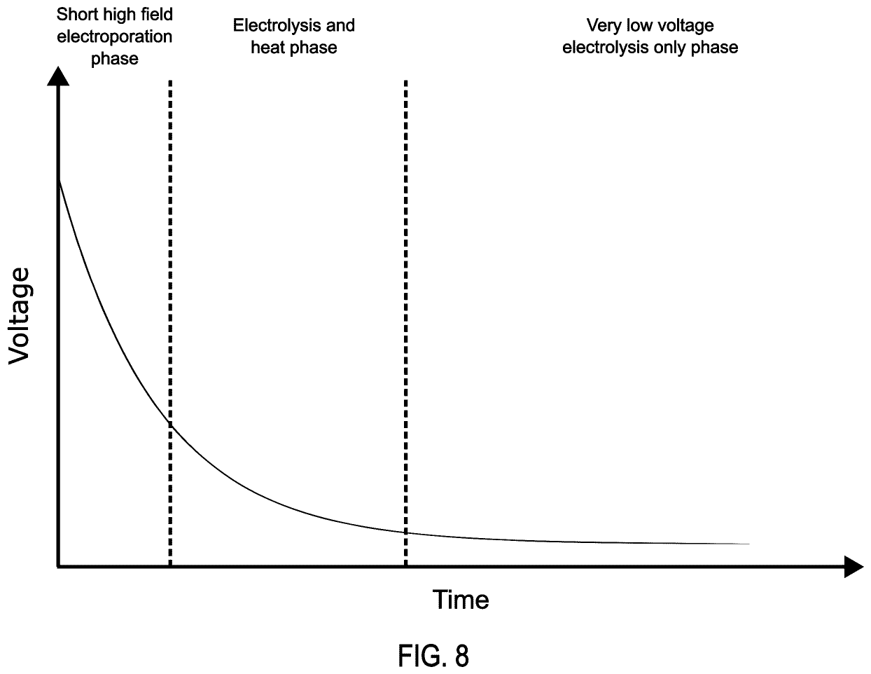

In case the permeabilization is performed by means of electroporation, all principal modes of ablation are based on electric energy (electrical fields in tissue) and require some kind of anode and cathode. In previous patents it was established how different pulse sequences can be combined to optimize ablation efficacy and safety by means of pulse shaping (REF) or modifying an exponential decay pulse (REF), which at the same time can lead to the three aforementioned ablation modes. FIG. 8 outlines how within a relatively short amount of time an E2 pulse based on a simple exponential decay (e.g. capacitor discharge) passes through an electroporation phase, electrolysis, and heat phase and towards the tail of the exponential decay a pure electrolysis phase, within the discharge of a single pulse.

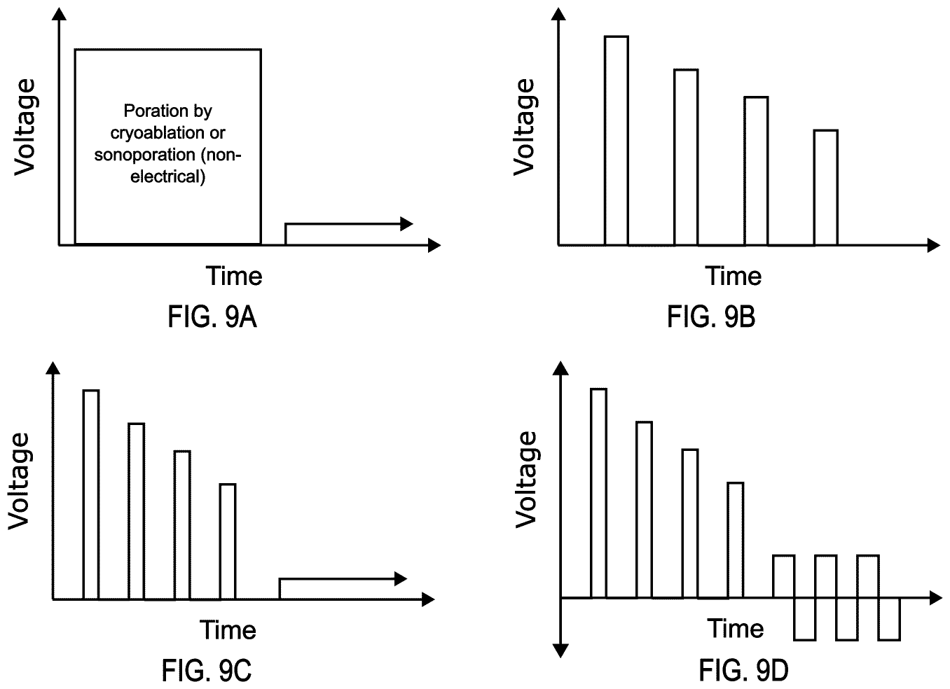

FIG. 9A-D shows how for no adjustment of electrode configuration, further immunological reactions can be targeted using the combination of different ablation modalities and through the use of the modulated waveforms specified in U.S. Patent No. US20210186592A1.

FIG. 9A outlines a pulse sequence where first, cell poration is achieved either by cryoablation or sonoporation, followed by a phase of electrolysis.

FIG. 9B outlines a pulse sequence where a pulse comparable to the one in FIG. D is chopped to increase field strength and maximize safety as specified in U.S. Patent No. US20210186592A1.

FIG. 9C-D outline the same chopped pulse from FIG. 9B followed by an electrolysis phase (no heat) and a heat phase by either microwave radiation or other (with no electrolysis), respectively.

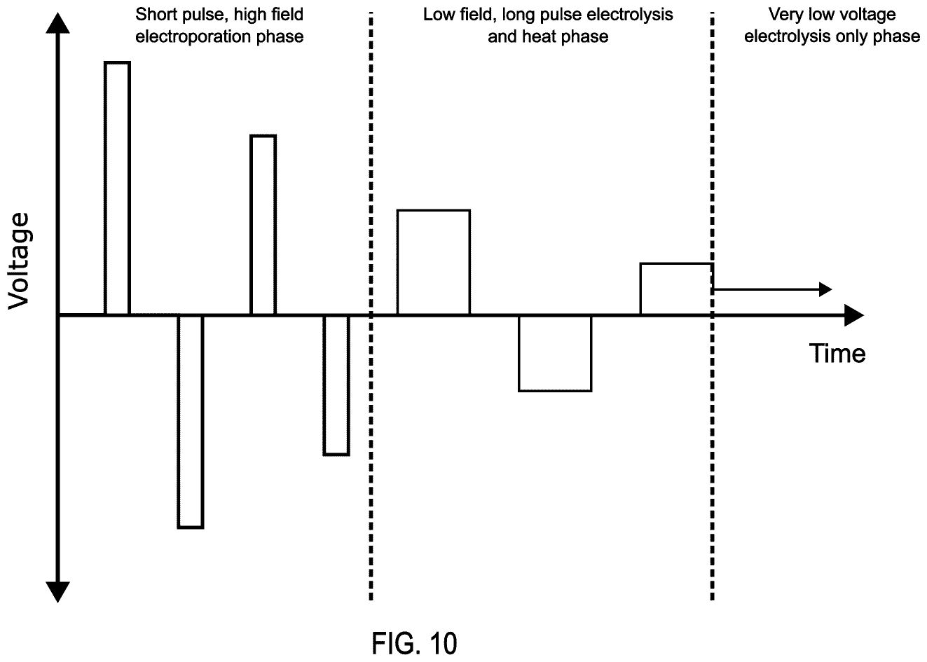

Finally, a combined sequence with bipolar pulses for safety as outlined in U.S. Patent No. US20210186592A1 consisting of electroporation, electrolysis, and heat application within one wave of typically few hundred milliseconds (FIG. 10).

The combination of these three distinct ablation modes appears both optimal for the purpose of inducing, optimizing, and specifying a wide range of responses due the inherently different response types that now can not only be combined but applied “at once” with one system and apparatus.

Considering the possible applicable parameters of the pulse(s) (energy intensity over time) and their ability to ablate by different cell death modes, in combination with the adjustability of electrode configurations (with some given as examples in this disclosure), one can create treatments that target certain individual, or even a combination of immunological responses.

Practically, this implies that with a knowledge of the electrode geometry (either by user input or impedance or time of flight sensors) and either an estimate or a measurement of the tissue resistance, a computer model can approximate solutions to the named differential equations for the norm of the electrical field, Penne's bioheat and Nernst Planck diffusion, through which the extent of certain immunological responses can be predicted.

Inversely, targeting a certain outcome, one can define the desired immune response(s) or cell death type(s) and have the system calculate the optimal parameters for achieving this for a certain tissue and applicator geometry.

Further computational optimization, considering the multi-variability of the system is limited by computational power and constraints, however the invention can be combined with many existing and developing techniques to obtain solutions for effective combinations of electrode configuration and pulse sequences which synergistically maximize the desired immunological outcomes.

Though there is no universal recipe by which an ablation process will cause the best immune response for each cancer type as cancer is highly inhomogeneous, the invention allows to optimize for each of the immune responses as per the oncologist or the medical oncologist, immunologist or molecular genetic pathologist—as well as to simply maximize for DAMP and cytokine release inside a volume of interest and hence maximize the subsequent macrophage invasion.

Without limiting the scope of the present invention to any specific choice of parameters or geometries, the following section details observed immunological reactions from experimental results obtained using different E2 pulse sequences via axial macroscopic pathological cuts in pig liver 24 h post ablation using two 1 mm needle type electrodes. This is solely to support the claims and show that by simply changing pulse parameters and thereby the ablation modes, the immunological response changes predictably.

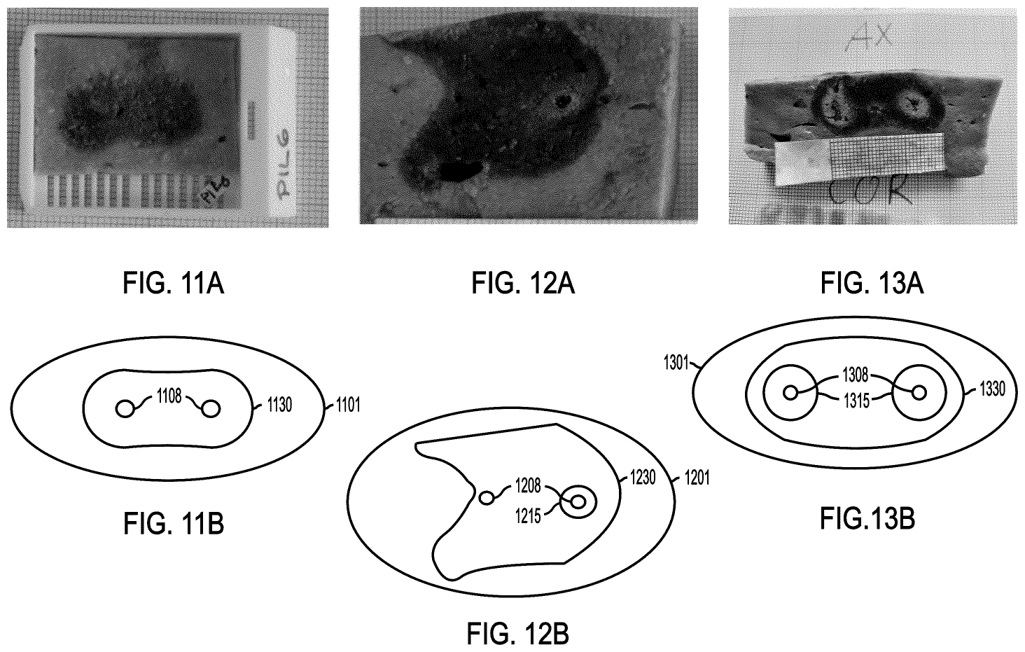

FIG. 11A demonstrates an image of a sample obtained with a pulse sequence with a low amount of total energy and which is heavily electroporation focused with little electrolysis and no thermal ablation effect. FIG. 11B shows that the tissue 1101 shows only electroporation associated cell death modes 1130 within it.

FIG. 12A demonstrates an image of a sample obtained with a pulse sequence with approximately double the energy and significantly more electrolysis than used for the sample in FIG. 11A. A small amount of thermal necrosis 1215 in addition to electroporation-based cell death modes 1230 is visible on the right electrode 1208 as outlined in FIG. 12B.

FIG. 13A demonstrates an image of a sample using relatively high energy amount in a very short time frame (few seconds). FIG. 13B outlines the associated cell death modes, whereby in close vicinity to the two electrodes 1308 this caused visible areas of thermal necrosis 1315 surrounded by areas dying though pyroptosis 1330 which is highly pro inflammatory.

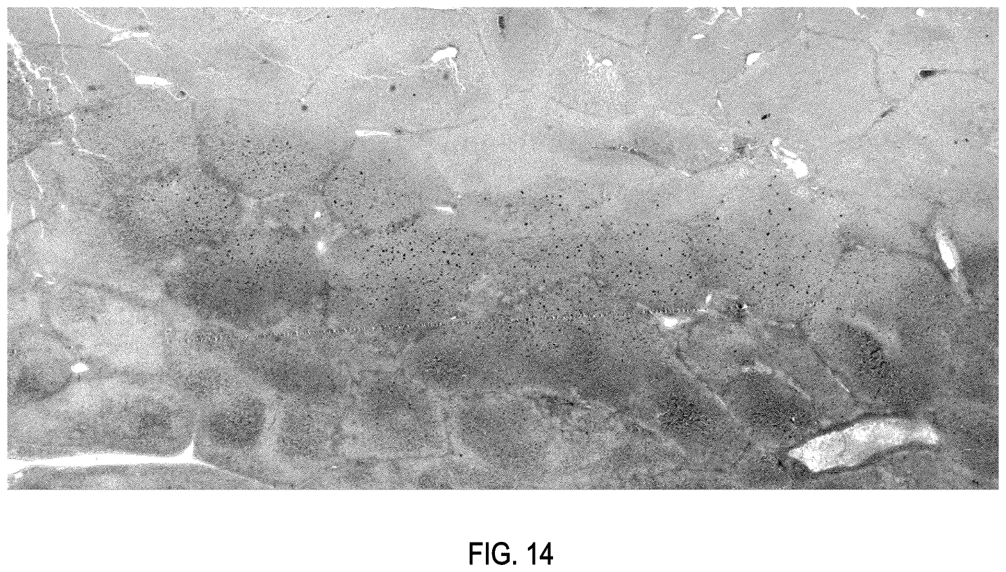

FIG. 14 represents an H&E stain at 20× magnification, of a healthy pig liver 24 h after E2 application with an electrode placement analogous to that of FIG. 13C. Whilst in the upper part of the image direct thermal damage with limited perfusion due to denaturation of the tissue and blood supply is seen, due to the proximity of the electrode, towards the bottom a very high macrophage concentration can be seen associated with the attraction of HSPs and the thermal necrosis and pryoptosis at play. Limiting the area of pure thermal necrosis makes the ablation site more permeable to the immune system's surveillance and response mechanisms, facilitating a targeted and potentially more efficacious immunological engagement.

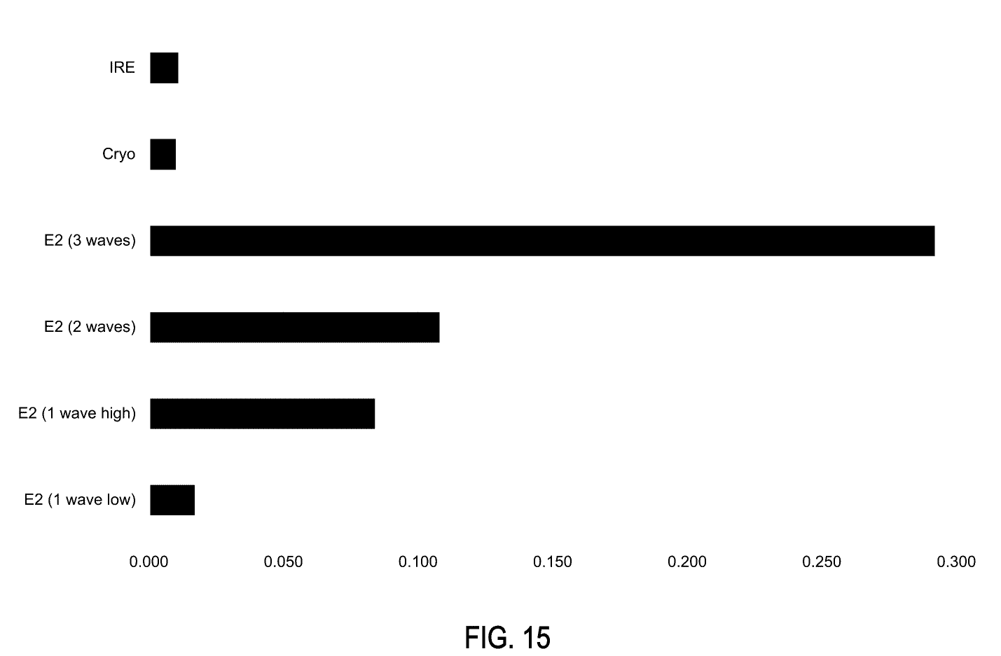

Fine tuning application mode and pulse sequence used can alter the number of macrophages present near the ablation site. FIG. 15 shows the relative area of macrophages in pig liver for different methods of energy delivery, roughly 24 hours after ablation and counted using standardized image analysis of high-resolution scans of pathological H&E stained slices of the ablated treatment sites. FIG. 15 demonstrates the exceptionally large quantities of macrophages following E2 treatments, when compared to cryoablation or standard IRE, especially when multiple higher energy waves are applied. More specifically the samples from the study showed that increasing the volume of tissue ablated by means of pryoptosis increases the immune response in terms of macrophage invasion to the ablated site. It also supports the claim that a simple change of parameters predictably changes the ablation modes and hence immune response.

As done in the example above, using for example computer models with Penne's Bioheat Equation, one can tune the E2 parameters to increase the macrophage invasion to certain areas of the ablation site by increasing the amount of cell death by pryoptosis close to area of interest.

Analog, one could optimize for cell death types typical to certain electrolytic species by using computer models solving Nernst Planck type diffusion, which describes the type of diffusion relevant to E2 in tissue.

Using the same computer methods as above, one can also choose the applicator type, shape and geometry and sequence.

Since the E2 system is capable of delivering flexible pulse sequences ranging from nanoseconds to many seconds and operates across a wide voltage spectrum, accommodating low voltages in the tens to hundreds of volts range and high voltages extending into the thousands, it can be configured with numerous connectors for various types of applicators, providing clinicians with the flexibility to select pulse sequences tailored for specific ablation strategies or immunological outcomes.

In some examples, the selected pulse sequences are optimized for different purposes. For instance, sequences may be focused on extensive ablation, including options for large, rapid, or vessel-preserving ablation. Alternatively, sequences can be designed to specifically generate DAMPs or to predominantly induce particular cell death modes such as pyroptosis, necroptosis, apoptosis, or necrosis.

In certain embodiments, the system integrates sensors that can include temperature, current, or impedance sensors, providing real-time feedback. This feedback is utilized by the computer system to assess the ablation process's status and efficacy, ensuring the achievement of the intended sequence goals and facilitating adjustments as needed.

In some examples, applicator designs incorporate multiple “hotspots,” such as needle matrices, to target the maximization of cell death types like pyroptosis and necrosis. These applicators aim to enhance the inflammatory response and the release of heat shock proteins while preventing the formation of large volumes of non-perfused tissue.

In certain embodiments, applicators are fashioned as very thin needles or arrays suitable for minimally invasive insertion and blood vessel penetration. These applicators are particularly effective for stimulating immune responses, rather than achieving complete tissue ablation, and may be used in conjunction with adjuvant immunotherapies.

In some examples, surgical or percutaneous applicators are employed to address inoperable tumor sections. These applicators are designed to induce localized immune reactions and can be augmented by additional immune therapies, such as small molecules, gene therapy vectors, cytotoxic substances, or cytokines administered by electroporation, further enhancing the therapeutic impact.

The preferred embodiment of the invention is described herein to illustrate a best mode of use, providing sufficient detail to enable one skilled in the art to make and use the invention. Consider a patient diagnosed with a multi-centimeter tumor located within an organ, such as the liver, where the tumor encases a major blood vessel. To initiate treatment, several monopolar needles are strategically placed around the tumor perimeter. This placement is achieved using standard radiological imaging techniques to ensure the vessel remains undisturbed. Once the needles are positioned, the system connects to the probes and generates a tailored treatment plan. First, the system measures distances between probes and tissue impedances between all probes (alternatively, these values can be set manually). Then the clinician with knowledge about the overall oncological treatment plan for the patient initiates the planning and setting process on the system for the volume of interest across different regions of and around the tumor based on their proximity to critical structures: peripheral tumor zones: At the tumor's edges, a high concentration of electrolysis is employed to disrupt the tumor microenvironment (TME), aimed at undermining the tumor's defensive barriers and enhancing the efficacy of subsequent treatments. vicinity of the vessel: close to the vessel, the treatment strategy restricts itself to electroporation-based ablation combined with minimal electrolysis (referred to as the E2 process). This approach ensures the integrity of the vessel is maintained while still targeting tumor cells for destruction. Central tumor mass: In the bulk of the tumor away from the vessel, targeted spots of thermal necrosis are introduced. This not only expands the treatment volume but also increases the production of damage-associated molecular patterns (DAMPs) and triggers acute inflammatory responses, which are crucial for an effective immunological assault on the tumor. Obvious to the skilled practitioner, these given preferred energy application examples would completely change if for example adjuvant check point inhibitors, interleukins, T-Cell injections or other immunomodulates were within the treatment plan for the patient. The system calculates and applies the appropriate pulse sequences, total energy, voltage, required cooling or heating times for each electrode pair to approximate the ablation distribution in accordance with the ablation plan for the volume; which consists of delivering precisely timed, precise amounts of electric energy to all electrodes in pairs of two or more electrodes per sequence/wave. Upon completion of the energy application, the immune response is evaluated over the next days and weeks using standard clinical methods. Based on this assessment, the treatment may be adjusted and reapplied, if necessary, to the same or additional tumorous sites within the body.

This best mode implementation leverages the system's capabilities to deliver a comprehensive, targeted, and minimally invasive treatment, tailored to complex tumor geometries and sensitive anatomical contexts. This approach not only maximizes therapeutic outcomes but also minimizes potential complications and enhances patient recovery.

In summary, the present disclosure builds upon U.S. Patent No. U.S. Patent No. US20210186592A1 and No. US20210330371A1 which detail how different pulse sequences and shaping can accomplish different ablation and safety effects. This invention now expands this to encompass optimization of the pulse sequences in regard to a desired immunological response.

The described invention is a system and apparatus that incorporates combinations of permeabilization (electroporation) and electrolysis and thermal ablation via one closed loop ablation process that can be optimized via an interface to yield an ablation effect and/or an immunological response in a volume of interest and furthermore be used with adjuvant or as an adjuvant therapy to other immunological therapies and cancer treatments in general.

Various non-limiting aspects include:

In a first aspect, a system for optimizing towards a desired immune response via different cell death modes in a volume of interest, comprising: a plurality of electrodes configured for delivering electroporation and electrolysis to target tissue; a thermal modulation unit integrated able to heat and/or cool and designed to adjust the temperature of the target tissue during ablation; a controller programmed to synchronize electroporation, electrolysis, and thermal inputs based either on real-time feedback from the tissue (impedance, temperature, or changes in electrical conductivity) or generic tissue data to induce a specified ablation mode or immune response in a volume(s) of interest; wherein the controller adjusts parameters including voltage, current, total charge, pulse modulation (chopping), and thermal energy to achieve desired immune outcomes through the ablation mode and in turn the modulation of cytokine release and DAMP activation in the volume(s) of interest; a user interface for clinicians to plan the treatment, input data, select parameters and monitor the treatment.

In a second aspect, a method for inducing a desired immune response in tissue (tumor) ablation procedures, comprising: planning the procedure by means of computational modeling potentially in conjunction with previously acquired tomography datasets and an immune-oncological treatment plan; resulting in a map of ablation types or cell death types (Pyroptosis, Necroptosis, Apoptosis, Necrosis, Ferroptosis) or immune responses (DAMP release, Cytokines release, Antigen presentation, Programmed lysis, delayed responses, Activation of cytotoxic T lymphocytes, Tumor Micro Environment (TME) disruption, T-cell activation, NK cell activation) desired for volume(s) of interest to achieve the planned immunological effect, applying permeabilization (including but not limited to electroporation) and electrolysis through a set of electrodes to a target tissue area with voltages and energies and pulse shapes that computationally approximate the planed ablation map of the volume(s) of interest, concurrently and optionally adding thermal energy (via electric pulse modulation) to the target tissue to enhance the effects of electroporation and electrolysis, and optionally adjusting the parameters of electroporation, electrolysis, and thermal energy dynamically, based on the sensor readings (impedance and temperature).

In a third aspect, an apparatus for tissue ablation designed to induce different ablation modes in a volume of interest in a controlled fashion consisting of: electrodes capable of delivering both electroporation and electrolysis and radiofrequency and microwave type pulse modulations; optionally the electrodes can be hollow to allow cooling of the volume of interest as well, a computing device with a user interface that allows the clinician to make plannings for the ablation volume that allows him to adequately set and/or assess what ablation types and/or immunological responses will occur at which place in the volume(s) of interest, a control system programmed to adjust the delivery parameters of electroporation, electrolysis, and heating/cooling based on the user input to induce specific types of cell death associated with beneficial immune reactions in a tissue volume of interest, a waveform generator able to apply adequate waveforms to achieve any combination of electroporation, electrolysis, electrolytic electroporation, heating for a range of electrode geometries, and means for measuring tissue temperature and impedance for estimating the ablation types that occurred with feedback to the user or the control system either before, during (real time adjustment) or after the initial ablation energy delivery.

A further aspect includes the second aspect, wherein the thermal energy is applied selectively based on the tissue type and the desired depth of ablation, and is controlled to prevent overheating while maximizing the release of heat shock proteins and other immunostimulatory DAMPs to planned areas using the described functions of the apparatus of aspect 3.

A further aspect includes the third aspect, where the permeabilization is not done by means of electroporation but by sonoporation or cryoablation or other means of cell permeabilization.

A further aspect includes the first aspect, where the electrode configuration includes options for both invasive (including but not limited to endoscope attachments like clamps or plates or tips; needle arrays; umbrellas; plates; internal strings or anchors) and non-invasive application (including but not limited to surface pads, needle pads, sponges, flexible electrode arrays, conductive gels), allowing for flexibility in treating both superficial and deep-seated tumors.

A further aspect includes the second aspect, further comprising the step of administering adjunctive immunotherapeutic agents concurrently with the ablation procedure to synergize with the induced immune responses. Immunotherapies potentially benefiting include but are not limited to: Checkpoint Inhibitors, Cancer Vaccines, Cytokine Therapy, Monoclonal Antibodies, Adoptive Cell Transfer, Oncolytic Virus Therapy, Cancer Growth Inhibitors, Bispecific Antibodies, Immune System Modulators, CAR T-Cell Therapy, Tumor-Infiltrating Lymphocytes (TIL) Therapy, Dendritic Cell Vaccines, Immune Agonists, Immune Adjuvants, Toll-like Receptor Agonists, Interferons, Interleukins.

A further aspect includes the second aspect with treatment combination and repetition timings adapted to the oncological and immunological response as measured by suitable imaging and blood tests, respectively.

1. A system for optimizing towards a desired immune response via different cell death modes in a volume of interest, comprising:
a) a plurality of electrodes configured for delivering electroporation and electrolysis to target tissue;
b) a thermal modulation unit integrated able to heat and/or cool and designed to adjust the temperature of the target tissue during ablation;
c) a controller programmed to synchronize electroporation, electrolysis, and thermal inputs based either on real-time feedback from the tissue or generic tissue data to induce a specified ablation mode or immune response in a volume(s) of interest; wherein the controller adjusts parameters including voltage, current, total charge, pulse modulation, and thermal energy to achieve desired immune outcomes through the ablation mode and in turn the modulation of cytokine release and DAMP activation in the volume(s) of interest; and
d) a user interface for clinicians to plan the treatment, input data, select parameters and monitor the treatment.

2. A method for inducing a desired immune response in tumor ablation procedures, comprising:
a) planning the procedure by means of computational modeling in conjunction with previously acquired tomography datasets and an immune-oncological treatment plan; resulting in a map of ablation types or cell death types including pyroptosis, necroptosis, apoptosis, necrosis, ferroptosis, or combinations thereof or immune responses including DAMP release, cytokine release, antigen presentation, programmed lysis, delayed responses, activation of cytotoxic T lymphocytes, tumor micro environment (TME) disruption, T-cell activation, NK cell activation, or combinations thereof desired for one or more volumes of interest to achieve the planned immunological effect;
b) applying permeabilization, including electroporation and electrolysis through a set of electrodes to a target tissue area with voltages and energies and pulse shapes that computationally approximate the planed ablation map of the volume(s) of interest;
c) concurrently adding thermal energy using electric pulse modulation to the target tissue to enhance the effects of electroporation and electrolysis;
d) adjusting the parameters of electroporation, electrolysis, and thermal energy dynamically, based on the sensor readings including impedance and temperature.

3. An apparatus for tissue ablation designed to induce different ablation modes in a volume of interest in a controlled fashion, the apparatus comprising:
a) electrodes capable of delivering both electroporation and electrolysis and radiofrequency and microwave type pulse modulations;
b) a computing device with a user interface that allows the clinician to make plannings for the ablation volume that allows him to adequately set and/or assess what ablation types and/or immunological responses will occur at which place in the volume(s) of interest;
c) a control system programmed to adjust the delivery parameters of electroporation, electrolysis, and heating/cooling based on the user input to induce specific types of cell death associated with beneficial immune reactions in a tissue volume of interest;
d) a waveform generator able to apply adequate waveforms to achieve any combination of electroporation, electrolysis, electrolytic electroporation, heating for a range of electrode geometries; and
e) means for measuring tissue temperature and impedance for estimating the ablation types that occurred with feedback to the user or the control system either before, during or after the initial ablation energy delivery.

4. The method of claim 2, wherein the thermal energy is applied selectively based on the tissue type and the desired depth of ablation, and is controlled to prevent overheating while maximizing the release of heat shock proteins and other immunostimulatory DAMPs to planned areas.

5. The apparatus of claim 3 where the permeabilization is not done by means of electroporation but by sonoporation or cryoablation or other means of cell permeabilization.

6. The system of claim 1, where the electrode configuration includes options for both invasive and non-invasive application, for flexibility in treating both superficial and deep-seated tumors.

7. The method of claim 2, further comprising the step of administering adjunctive immunotherapeutic agents concurrently with the ablation procedure to synergize with the induced immune responses, including checkpoint inhibitors, cancer vaccines, cell fragment binding and presenting compounds, cytokine therapy, monoclonal antibodies, adoptive cell transfer, oncolytic virus therapy, cancer growth inhibitors, bispecific antibodies, immune system modulators, CAR T-Cell therapy, tumor-infiltrating lymphocytes (TIL) therapy, dendritic cell vaccines, immune agonists, immune adjuvants, toll-like receptor agonists, interferons, interleukins, or combinations thereof.

8. The method of claim 2 including treatment combination and repetition timings adapted to the oncological and immunological response as measured by suitable imaging and blood tests, respectively.
